(19) United States
(12) Patent Application Publication
(10) Pub. No.: US 2023/0310189 A1
(43) Pub. Date: Oct. 5, 2023

(54) REINFORCED SHEATH AND WIRE ASSEMBLY AND VASCULAR INTERVENTION DEVICE DELIVERY SYSTEM USING SAME

(71) Applicant: Cook Medical Technologies LLC, Bloomington, IN (US)

(72) Inventors: Joe Dervan, Portumna (IE); Brendan Flanagan, Macroom (IE)

(21) Appl. No.: 18/118,805

(22) Filed: Mar. 8, 2023

Related U.S. Application Data
(60) Provisional application No. 63/324,843, filed on Mar. 29, 2022.

(51) Int. Cl. A61F 2/966

(52) U.S. Cl. CPC A61F 2/966; A61F 2002/9623

(57) ABSTRACT

A reinforced cardiovascular sheath and wire assembly includes an inner tube with an external surface extending between a proximal end and a distal end. A reinforcement braid is in contact with the external surface of the inner tube. A wire with a distal segment is interwoven into, and in contact with, the reinforcement braid. A proximal segment of the wire extends proximally from the proximal end of the inner tube out of contact with the reinforcement braid. An outer tube receives, and is in contact with, the reinforcement braid and the inner tube. A vascular intervention device delivery system utilizes the sheath/wire assembly as a retractable sheath and pull wire.

### TECHNICAL FIELD

The present disclosure relates generally to reinforced sheath/wire assemblies and also to vascular intervention device delivery systems that utilize such an assembly.

### BACKGROUND

Self expanding stents and similar vascular intervention devices are often delivered and deployed using vascular intervention device delivery systems. In the past, these systems were often identified as pin and pull systems. More recently, thumbwheel stent delivery systems have seen considerable success. This success has led to a demand for ever smaller stent delivery systems that can reach more remote locations, such as below the knee, which may require a system of 5 Fr. or less.

One area that contributes to overall diameter of stent delivery systems is the welded attachment between a pull wire and a retractable sheath. While the attachment strategy described in co-owned U.S. Pat. No. 10,327,927 has performed magnificently for years, the strategy has proven difficult to employ in smaller systems with a diameter of 5 Fr. or less.

The present disclosure is directed toward one or more of the problems set forth above.

### SUMMARY OF THE DISCLOSURE

In one aspect, a reinforced cardiovascular sheath and wire assembly includes an inner tube with an external surface extending between a proximal end and a distal end. A reinforcement braid is in contact with the external surface of the inner tube. A wire with a distal segment is interwoven into, and in contact with, the reinforcement braid. A proximal segment of the wire extends proximally from the proximal end of the inner tube out of contact with the reinforcement braid. An outer tube receives, and is in contact with, the reinforcement braid and the inner tube.

In another aspect, a vascular intervention device delivery system includes a catheter with a proximal end attached to a handle, and a distal carrier segment for mounting a vascular intervention device thereon, and defines a longitudinal axis. A retractable sheath includes a reinforcement braid and is movable from a first position covering the distal carrier segment to a second position retracted proximally uncovering the distal carrier segment. A pull wire is mechanically attached to, and in contact with the reinforcement braid of, the retractable sheath and extends proximally from the retractable sheath toward the handle.

### BRIEF DESCRIPTION OF THE DRAWINGS

FIG. 1 is a perspective schematic view of a vascular intervention device delivery system according to the present disclosure;

FIG. 2 is an enlarged view of the distal segment of the delivery system shown outlined with a dashed line in FIG. 1;

FIG. 3 is a view similar to FIG. 2 about half way through a deployment of a self expanding stent;

FIG. 4 is a sectioned side view of a handle portion of a vascular intervention device delivery system according to the present disclosure;

FIG. 5 is a top view of the inner workings of the vascular intervention device delivery system of FIG. 4, minus the handle;

FIG. 6 is a sectioned view through a portion of the vascular intervention device delivery system as viewed along section lines 6-6 of FIG. 5;

FIG. 7 is a side view of the pull wire partially wound onto a spool for the thumbwheel of FIG. 4;

FIG. 8 is a perspective schematic view of an attachment region of the pull wire to the retractable sheath according to one aspect of the present disclosure;

FIG. 9 is a perspective schematic view of the attachment region of a pull wire to the retractable sheath according to another aspect of the present disclosure;

FIG. 10 is a top view of a pull wire according to the present disclosure;

FIG. 11 is a sectioned view through the pull wire of FIG. 10 as viewed along section line 11-11;

FIG. 12 is a perspective view of a wire interwoven into a reinforcement as part of a sheath/wire assembly according to the present disclosure; and

FIG. 13 is a sectioned view of a sheath/wire assembly as viewed along section lines 12-12 of FIG. 1.

### DETAILED DESCRIPTION

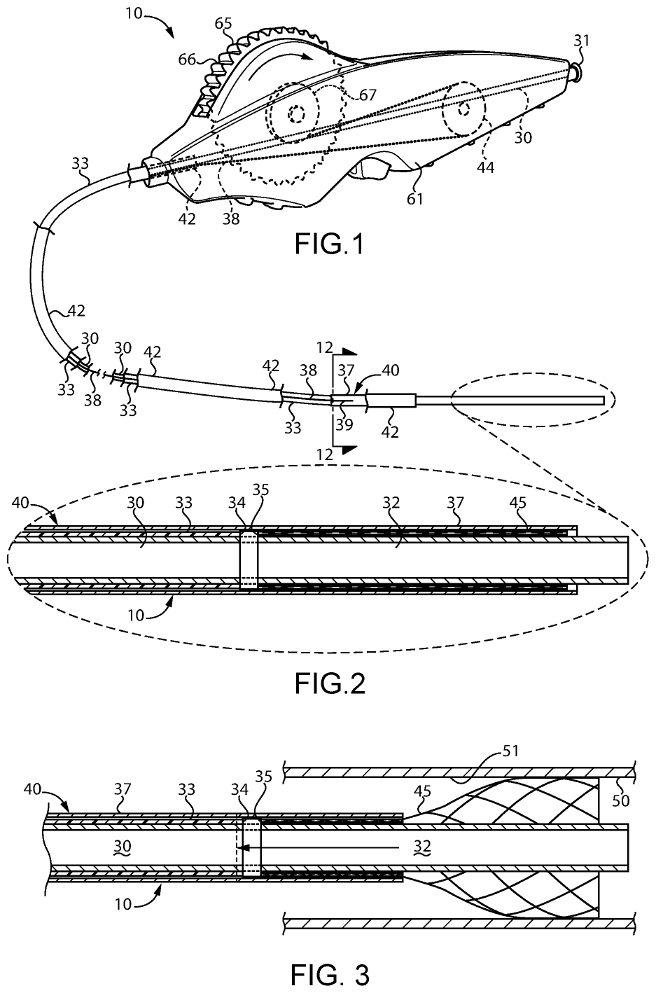

Referring initially to FIGS. 1-3, a vascular intervention device delivery system 10 is shown before and during delivery of a self expanding stent 45 into the vessel 50 of a patient. Delivery system 10 includes a handle 61 that may be gripped in one hand by a user during a delivery procedure. Handle 61 may, for instance, be manufactured from a suitable molded plastic, such as in two longitudinal halves that are joined in any suitable manner, such as via a mechanical connection, to form the complete handle 61. A thumbwheel 65 is rotatably mounted in the handle 61 and has a radially outward thumb surface 66 and a spool 67. An inner catheter 30 has a proximal end 31 attached to handle 61, and a distal carrier segment 32 for mounting a vascular intervention device, such as a self expanding stent 45, thereon. Proximal end 31 may take the form a Luer lock fitting to receive a wire guide, or so that treatment fluids or the like may be injected through inner catheter 30 in a manner well known in the art. The Luer lock fitting that comprises the proximal end 31 of inner catheter 30 may be mechanically connected to the handle, such as via an interaction between molded surfaces of the handle halves and the external surface of the Luer lock fitting. A retractable sheath 37 is movable with respect to inner catheter 30 from a first position covering the distal carrier segment 32 to a second position indicated by the dashed line in FIG. 3 at which the retractable sheath 37 has been retracted proximally to uncover the distal carrier segment 32. FIG. 3 shows the retractable sheath 37 about half way between the first position and the second position. Delivery system 10 may include an outer catheter 33 with a distal end 34 that terminates as a pusher band at a proximal end 35 of the distal carrier segment 32 in a manner similar to other stent delivery systems known in the art. The distal end 34 of outer catheter 33 may assist in preventing movement of stent 45 in a proximal direction when retractable sheath 37 is being slid from its first position toward its second position. A proximal end of the outer catheter 33 may be positioned in, and attached to, handle 61.

A pull wire 38 extends between the spool 67 of thumbwheel 65 and the retractable sheath 37. Pull wire 38 and retractable sheath 37 comprise a sheath/wire assembly 40 according to the present disclosure. Pull wire 38, which preferably is less elastic than the retractable sheath 37, may be attached to retractable sheath 37 at an attachment 39, which may include having the pull wire 38 interwoven into a metallic reinforcement braid of retractable sheath 37. In all cases of this disclosure, the attachment 39 is a mechanical connection that does not include a weld. In some versions of the vascular intervention device delivery system 10 of the present disclosure, pull wire 38 will be longer than retractable sheath 37. Nevertheless, retractable sheath 37 could be longer than pull wire 38 without departing from the present disclosure. Pull wire 38 may comprise a metallic curved cross-section thin band of metal, such as spring steel.

A wire retention/stability sheath 42 receives and surrounds a majority of the length of pull wire 38, and serves to keep pull wire 38 in close proximity to the outer surface of outer catheter 33 over much of the length of delivery system 10. Stability sheath 42 may receive at least a portion of each of the pull wire 38, the inner catheter 30 and the outer catheter 33, as shown. In the illustrated embodiment, wire retention/stability sheath 42 terminates, and is attached at its proximal end, at a fixation point within handle 61. Nevertheless, other configurations for attachment of wire retention/stability sheath 42 would also fall within the intended scope of this disclosure.

When in its pre-deployment configuration, as shown in FIGS. 1 and 2, a vascular intervention device, such as a self expanding stent 45, is disposed between an outer surface of the distal carrier segment 32 of inner catheter 30, and an inner surface of the retractable sheath 37. In the illustrated example, stent 45 may be a self expanding 5 Fr. stent. During a typical procedure, the distal carrier segment 32 is positioned at a treatment location 51 within a vessel 50 of a patient, which may be below the patient's knee. After achieving proper positioning, the user then grips handle 61 and begins to rotate thumbwheel 65 so that pull wire 38 is wound onto spool 67. As this occurs, pull wire 38 and retractable sheath 37 move proximally with respect to inner catheter 30 to allow the self expanding stent 45 to expand away from carrier segment 32 and into contact with the inner wall of vessel 50 in a manner well known in the art. During this process, inner catheter 30 and outer catheter 33 may be placed in compression while the sheath/wire assembly 40 (pull wire 38 and retractable sheath 37) is in tension. According to the present disclosure, handle 61 and thumbwheel 65 may include a structure that allows thumbwheel 65 to rotate to wind pull wire 38 onto spool 67, but prevent rotation in an opposite direction through a ratchet structure within the handle 61. This aspect of the disclosure allows the user to stop the deployment procedure while retaining the stored elastic energy in pull wire 38 and retractable sheath 37.

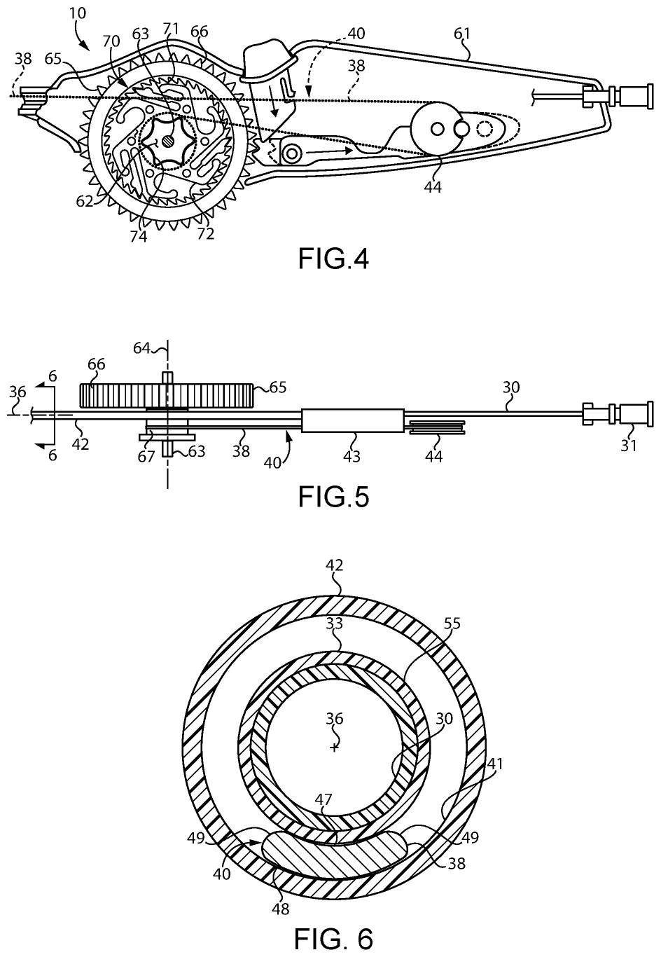

Referring now in addition to FIGS. 4-13, various features of the vascular intervention device delivery system 10 are shown and discussed. Handle 61 may be formed from a suitable plastic to include a star shaped hub 62 that is received in a matching star shaped opening 74 defined by ratchet pawl 72 of a ratchet 70. This configuration permits assembly of ratchet pawl 72 fixed against rotation to star shaped hub 62 in a plurality of different but equivalent angular orientations. Star shaped hub 62 may define a central opening that receives an axle 63 to define an axis 64 about which thumbwheel 65 rotates. Thumbwheel 65 includes a radially outward thumb surface 66 and a radially inward ratchet surface 71 of the ratchet 70. Thumbwheel 65 may also include a spool 67 upon which the pull wire 38 is wound when the device delivery system 10 is operated. In this version, the wire retention/stability sheath 42 terminates at a junction box 43 (not shown in FIG. 4 for the sake of clarity) positioned within handle 61. The pull wire 38 is positioned within the wire retention/stability sheath 42 and emerges from the junction box 43 to wrap around an idler wheel 44 and return in a reverse direction for being wound onto spool 67. Idler wheel 44 is rotatably mounted in handle 61 and positioned proximal to thumbwheel 65. Ratchet 70 prevents thumbwheel 65 from rotating in a forward direction, but the retractable sheath 37 (FIGS. 1-3) moves responsive to rotation of thumbwheel 65 in a reverse direction.

Referring now specifically to FIGS. 9-13, Pull wire 38 may be manufactured from a single length of spring stainless steel having the cross sectional shape best shown in FIG. 11. After being cut to length, some material may be removed to form a distal attachment segment 21 that is separated from a majority segment 19 by a tapered transition segment 22. This removal of material may be accomplished by, for instance, a punch and dye process, or by some machining process such as electrical discharge machining in a manner well known in the art. As best shown in FIG. 11, the distal attachment segment 21 may have a cross sectional area that is smaller than a cross sectional area of the majority segment 19. The majority segment 19 may have a width 103 that is greater than a thickness 104. Likewise, the distal attachment segment 21 may have a width 105 that is greater than a thickness 109. Those skilled in the art will appreciate that majority segment 19 constitutes a majority of the length of pull wire 38. Thus, distal attachment segment 21 inherently constitutes a minority of the length of pull wire 38. As best shown in FIG. 11, the distal attachment segment 21 is flatter than the majority segment 19, in order to better facilitate attachment to retractable sheath 37. In the present example embodiment, “flatter” means that distal attachment segment occupies a smaller arc of a circle than the arc of the circle associated with the majority segment. In some versions, no material is removed from the distal segment of the pull wire 38 so that the entire length of the pull wire 38 has an identical cross section.

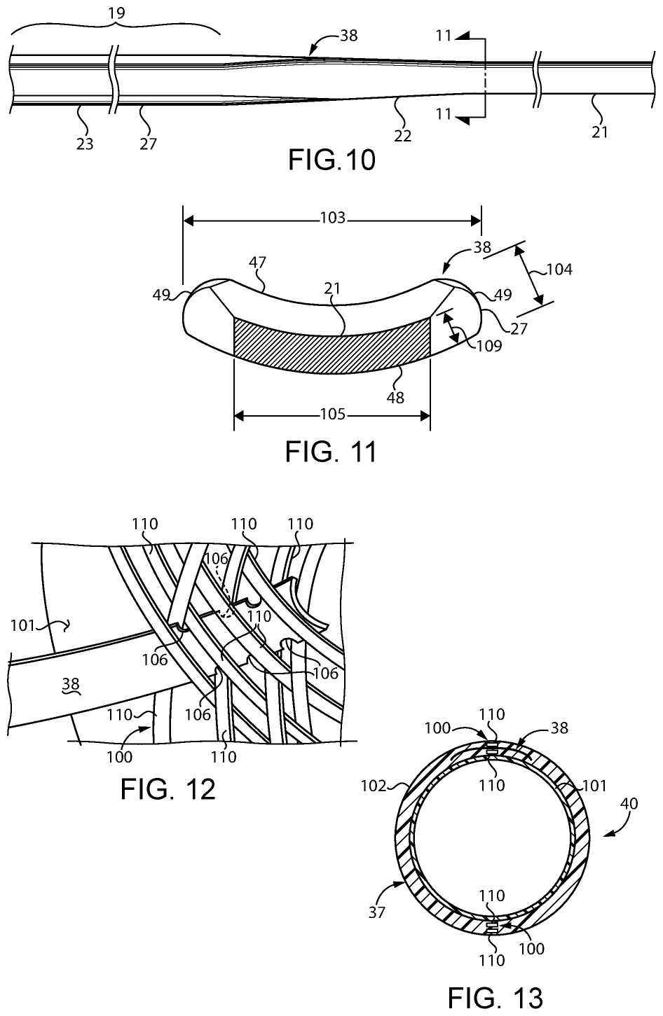

After pull wire 38 is machined or formed into the shape shown in FIGS. 10 and 11, the distal attachment segment 21 may be mechanically attached to retractable sheath 37. For instance, retractable sheath 37 always includes a reinforcement that may be a conventional metallic braid 100 that is interwoven with distal attachment segment 21. This construction keeps braid 100 and distal attachment segment 21 of pull wire 38 in place over an inner tube 101 of retractable sheath 37, which may be formed of a low friction polytetrafluoroethylene (PTFE). Then, an outer tube 102 of a suitable polymer jacket is created so that the braid 100 and the distal attachment segment 21 of pull wire 38 are laminated between the PTFE inner tube 101 and the polymer outer tube 102. Polymer jacket 102 may be applied in a conventional extrusion process. FIG. 12 is interest for schematically showing that strands of the braid preferably go both over and under pull wire. FIG. 12 is also of interest for showing that pull wire 38 may define side notches that each receive a respective strand of the reinforcement braid 100. FIG. 13 also shows strands of the braid positioned both over and under pull wire 38, with the polymer jacket outer tube 102 filling in all the spaces created by the pull wire 38 and braid 100 to better facilitate a secure connection for the sheath/wire assembly 40. FIG. 8 shows an attachment structure that may be suitable for shorter stents, such as those that may range from 40-120 millimeters in length. For longer stents, an attachment configuration like that shown in FIG. 9 may be substituted in place. In this version, the attachment is much the same as that of FIG. 8, except that the outer polymer cover tube 102 covers a segment 107 of pull wire 38 that is proximal to a proximal end 108 of the reinforement braid 100, which terminates at a location similar to that shown in FIG. 8. The bond is enhanced by laminating segment 107 of pull wire 38 between PTFE line tube 101 and polymerouter tube 102. The combination mechanical attachment 39 that includes interweaving the pull wire 38 into the braid 100 and applying a polymer jacket outer tube to laminate the segment 107 between the inner PTFE liner tube and the outer tube 102 will result in a more consistent, repeatable and lower profile joint than the welding strategy taught in the prior art. Much of the tension load during deployment is carried by being laminated with braid 100 and pull wire 38 between the inner PTFE liner tube 101 and the outer polymer cover tube 102. Thus, in the illustrated embodiment, retractable sheath 37 includes the inner PTFE liner tube 101, the braid or reinforcement 100 and the outer polymer cover 102. While the distal carrier segment 32 is being maneuvered to the delivery site 51, friction on the outer surface of retractable sheath 37 that might otherwise prematurely slide it toward its second position, is prevented at least in part by supporting a compression load in pull wire 38. The curved cross section of pull wire 38 helps to prevent pull wire 38 from buckling under this compression load, and the contact with pin 88 facilitates transfer of this compression load from the pull wire 38 to pin 88 and hence to handle 61 during the maneuvering step.

Referring specifically to FIGS. 6 and 11, pull wire 38 may have a curved cross section with a concave side 47 that is opposite to a convex side 48, both of which are flanked by rounded edges 49. A rounded edge according to the present disclosure includes a pair of radiused corners separated by a planar portion. The concave side 47 and the convex side 48 are the long sides of the pull wire 38 cross section. The concave side 47 faces longitudinal axis 36, and the convex side 48 faces away from longitudinal axis 36. In the illustrated embodiment, the idler wheel 44 and the spool 67 are arranged so that the convex side 48 is in contact with idler wheel 48, and the concave side 47 is in contact with spool 67. Nevertheless, those skilled in the art will appreciate that the opposite configuration would also fall within the scope of the present disclosure. Pull wire 38 may be positioned between stability sheath 42 and outer catheter 33. As best shown in FIG. 6, the convex side 48 of pull wire 38 is in contact with the inner surface 41 of stability sheath 42. The convex side 47 may be in contact with, or adjacent to, the outer surface 55 of outer catheter 33. In order to reduce friction, and reduce the contact area between pull wire 38 and stability sheath 42 as well as outer catheter 33, the radius of convex side 48 may be smaller than the radius of the inner surface 41 of stability sheath 42. Likewise, the radius of concave side 47 may be less than the outer radius 55 of outer catheter 33. Pull wire 38 may be manufactured from a suitable band of spring stainless steel to have the curved cross sectional shape shown in FIG. 6 (and FIG. 11). Pull wire 38 may be made from stainless steel with a sufficiently large cross section that the pull wire does not stretch when in tension at expected magnitudes (tens of Newtons) during a delivery process. The curved cross sectional shape of pull wire 38 may provide columnar support to retractable sheath 37 when in compression while the distal carrier segment 32 is being maneuvered to delivery site 51.

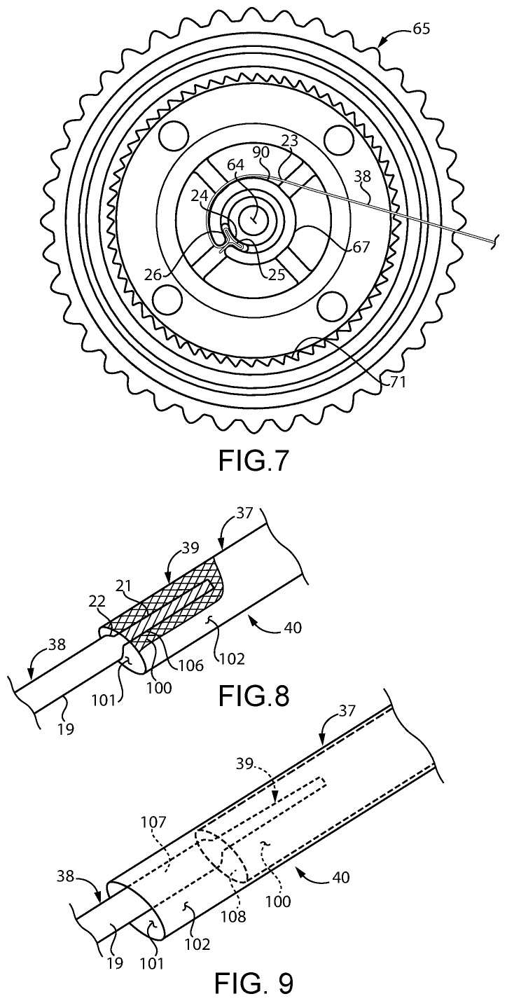

Referring now more specifically to FIG. 7, pull wire 38 is again shown in the form of a relatively thin band of spring steel with a curved cross section. Preferably, pull wire 38 is one integral length from its distal attachment point 39 to a proximal end 24 that is connected to spool 37. Pull wire 38 may have a majority of its length biased toward a straight configuration. In order to form proximal end 24 with an integral anchor 25, a proximal segment 23 of pull wire 38 may be annealed to provide greater ductility than a distal segment 27, which may comprise a majority of the length of pull wire 38. As used in the present disclosure, the term “integral” means that the identified features originate from the same part. Thus, integral anchor 25 is merely a deformed segment of the pull wire 38 and was never detached therefrom. An integral anchor 25 according to the present disclosure has never been separated from a remaining portion of pull wire 38, and then attached by some means such as a weld or adhesives or the like. Although integral anchor 25 could be formed on an un-annealed proximal segment 23 of pull wire 38, there are at least two reasons to consider annealing proximal segment 23 in order to increase ductility relative to the distal segment 27. First, forming un-annealed spring steel into the profile shape (T-shape) 26 can potentially result in breakage or substantial cracking at the severe bends where the wire band is bent back upon itself. Secondly, the best results have been observed when the vascular intervention device delivery system 10 is initially manufactured, stored prior to use, and during an initial use maneuvering to the desired implantation site by having the pull wire 38 wound at least one time and maybe as many as three to four times around the collection surface 90 of spool 67. In other words, proximal segment 23 may be wound at least once completely around collection surface 90 when the retractable sheath is at its first position covering the self expanding stent 45. The greater ductility of the proximal section 23 not only helps in the forming of the integral anchor 25 without cracking or breakage, but also better facilitates the initial winding of pull wire 38 onto spool 67.

In the illustrated embodiment, one might anneal a proximal segment 23 on the order of 30-40 millimeters in length, and form the integral anchor 25 out of maybe 10-15 millimeters of that proximal segment 23. The remaining portion of the proximal segment 23, and maybe some of the distal segment 27 may be wound onto spool 67 at the time of assembly and manufacture when retractable sheath 37 is still at its distal first position. By manufacturing with the expectation that at least one and maybe as many as three or four windings will begin on spool 67 when retractable sheath 67 is still in its first position, tight tolerances on a precise length for pull wire 38 are not necessary. Furthermore, tight tolerances with regard to what length of the pull wire 38 is consumed in order to form integral anchor 25 are also relaxed because of the initial windings on spool 67. This relaxation of dimensional length tolerances with regard to pull wire 38 not only reduces potential scrap, but also provides for a more robust design that arrives ready for use with little to no slack in pull wire 38 when the deployment procedure begins.

### INDUSTRIAL APPLICABILITY

The present disclosure is generally applicable to vascular intervention device delivery systems, and more particularly to a delivery system for delivery of self expanding stents and other vascular intervention devices with self expanding action. The present disclosure finds specific applicability to delivery of relatively long vascular intervention devices that produce substantial friction on the inner surface of retractable sheath 37, and thus require higher forces on retractable sheath 37 and pull wire 38 in order to successfully deliver the vascular intervention device to an intended treatment site. Finally, the present disclosure finds application to stent delivery systems for below the knee implant 5 Fr. stents. The sheath/wire assembly 40 of this disclosure might also find application in other medical devices apart from stent delivery systems.

Preferably but not necessarily, the sheath/wire assembly 40 may be made while the reinforcement is being braided onto the underlying PTFE liner tube in a so called braiding machine. For instance, a sacrificial wire might be welded to a distal end of the pull wire and then fed through an eyelet surrounding the braiding strands on the braiding machine. In order to ensure the pull wire 38 is interwoven straight, a weight might be utilized to keep the sacrificial wire and pull wire in tension during braiding until the distal end of the pull wire is interwoven into the braid. Thereafter, the sacrificial wire may be disconnected from the pull wire, and the remaining distal portion of the retractable sheath may then be braided. The outer polymer jacket may be applied before or after the underlying PTFE liner tube and braid are cut to define the proximal end of the retractable sheath. While the braid strands encircle the PTFE liner tube, the pull wire remains parallel to the axis of the PTFE liner tube where interwoven and where the pull wire extends away from the sheath 37. Although the interweaving results in braid strands under and over the pull wire 38 in the illustrated embodiment, the distal segment of the pull wire could be entirely underneath the braid without departing from the contemplated scope of this disclosure.

A method of operating vascular intervention device delivery system 10 includes rotating the thumbwheel 65 in a reverse direction to wind pull wire 38 onto spool 67 to build up tension in the retractable sheath 37 and pull wire 38 without moving the retractable sheath 37 relative to the distal carrier segment 32 of inner catheter 30. The “reverse direction” is clockwise in the view of FIG. 1 and counterclockwise in the view of FIG. 4. In both cases, the “reverse direction” means that the user's thumb moves toward their palm to rotate the thumbwheel 65. Next, a portion, which is less than all, of the distal carrier segment 32 may be uncovered by continuing to rotate the thumbwheel 65 in the reverse direction. During this portion of the procedure, friction between the pull wire 38 and the stability sheath 42 is limited at least in part by making a radius of the convex side 48 smaller than the inner radius 41 of stability sheath 42 as best shown in FIG. 6. Friction is limited by reducing the contact area between the two components. In addition, friction may also be limited by making a radius of the concave side 47 of pull wire 38 smaller than an outer radius 55 of outer catheter 33.

By forming pull wire 38 to have a relatively thin curved cross section, more material is available in the cross sectional shape so that the length of the pull wire 38 remains constant at tension levels (tens of Newtons), sufficient to move the retractable sheath 37 for its first position to its second position. Thus, while maneuvering to the delivery site, the pull wire 38 may be placed in compression to support a compression load, and during the delivery procedure the pull wire 38 is placed in tension to support delivery of the stent 45 to the delivery site 51. At some point during the delivery procedure, the user may then pause rotation of the thumbwheel 65 in the reverse direction. For instance, the user may pause in order to confirm that the vascular intervention device, such as a self expanding stent 45, is being delivered to the desired location in the vessel 50 of the patient. While the rotation of the thumbwheel 65 is paused, tension in the pull wire 38 and the retractable sheath 37 is maintained by holding the ratchet 70 and preventing rotation of the thumbwheel 65 in the forward direction. Ratchet 70 may be considered to be in a hold configuration when catches 73 are received in one of the stops 75 of the ratchet surface 71. A remaining portion of the distal carrier segment 32 is then uncovered to facilitate complete deployment of the self expanding stent 45 by resuming rotation of the thumbwheel 65 in the reverse direction until retractable sheath 37 arrives at its second position fully uncovering distal carrier segment 32.

It should be understood that the above description is intended for illustrative purposes only, and is not intended to limit the scope of the present disclosure in any way. For example, although the present disclosure is illustrated in the context of a thumb wheel actuated delivery system, those skilled in the art will appreciate that the pull wire of the present disclosure could find equal application in other delivery systems, such as pin and pull systems known in the art without departing from the present disclosure. Thus, those skilled in the art will appreciate that other aspects of the disclosure can be obtained from a study of the drawings, the disclosure and the appended claims.

What is claimed is:

1. A reinforced cardiovascular sheath and wire assembly comprising:
an inner tube with an external surface extending between a proximal end and a distal end;
a reinforcement braid in contact with the external surface of the inner tube;
a wire with a distal segment interwoven into, and in contact with, the reinforcement braid, and a proximal segment extending proximally from the proximal end of the inner tube out of contact with the reinforcement braid; and
an outer tube receiving, and in contact with, the reinforcement braid and the inner tube.

2. The assembly of claim 1 wherein the inner tube has length that is about equal to a length of the outer tube; and
the reinforcement braid has length that is shorter than both the inner tube and the outer tube.

3. The assembly of claim 1 wherein the proximal segment of the wire is longer than the distal segment.

4. The assembly of claim 1 wherein the inner tube is PTFE;
the outer tube is a polymer jacket; and
the wire is metallic.

5. The assembly of claim 1 wherein the inner tube has length that is longer than the distal segment of the wire.

6. The assembly of claim 1 wherein the wire has a cross section with a width that is greater than a thickness.

7. The assembly of claim 1 wherein the wire has a curved cross section with a concave side that is opposite to a convex side.

8. The assembly of claim 1 wherein a proximal segment of the wire extends proximally beyond the proximal end of the reinforcement braid, and is laminated between the inner tube and the outer tube.

9. The assembly of claim 1 wherein the distal segment of the wire includes a plurality of side notches that each receive a respective strand of the reinforcement braid.

10. The assembly of claim 1 wherein different strand segments of the reinforcement braid are in contact with an inner surface and an outer surface, respectively, of the distal segment of the wire.

11. The assembly of claim 1 wherein the distal segment of the wire has a curved cross section with a curvature that matches a curvature of the the reinforcement braid.

12. A vascular intervention device delivery system comprising:
a handle;
a catheter with a proximal end attached to the handle, and a distal carrier segment for mounting a vascular intervention device thereon, and defining a longitudinal axis;
a retractable sheath that includes a reinforcement braid and is movable from a first position covering the distal carrier segment to a second position retracted proximally uncovering the distal carrier segment;
a pull wire mechanically attached to, and in contact with the reinforcement braid of, the retractable sheath and extending proximally from the retractable sheath toward the handle.

13. The vascular intervention device delivery system of claim 12 wherein a distal segment of the pull wire is interwoven into the reinforcement braid.

14. The vascular intervention delivery system of claim 12 including a stability sheath that receives at least a portion of each of the pull wire, the catheter and retractable sheath; and
a 5 Fr. self expanding stent mounted about the distal carrier segment of the catheter.

15. The vascular intervention delivery system of claim 12 wherein reinforcement braid is laminated between an inner PTFE liner tube and an outer polymer tube, and the reinforcement braid is metallic; and
the pull wire is metallic.

16. The vascular intervention delivery system of claim 12 including a thumbwheel rotatably mounted in the handle and connected to a proximal end of the pull wire.

17. The vascular intervention delivery system of claim 12 wherein the pull wire has a curved cross section with a curvature that matches a curvature of the reinforcement braid.

18. The vascular intervention delivery system of claim 12 wherein a proximal segment of a polymer tube of the retractable sheath covers a segment of the pull wire that is proximal to a proximal end of the reinforcement braid.

19. The vascular intervention delivery system of claim 12 wherein a distal segment of the pull wire includes a plurality of side notches that each receive a respective strand of the reinforcement braid.

20. The vascular intervention delivery system of claim 12 wherein different strand segments of the reinforcement braid are in contact with an inner surface and an outer surface, respectively, of a distal segment of the pull wire.
